(19) United States
(12) Patent Application Publication
(10) Pub. No.: US 2016/0313349 A1
(43) Pub. Date: Oct. 27, 2016

(54) METHOD OF DIAGNOSIS AND TREATMENT

(71) Applicant: PARANTA BIOSCIENCES LIMITED, Caulfield, Victoria (AU)

(72) Inventor: David De Kretser, Surrey Hills, Victoria (AU)

(73) Assignee: PARANTA BIOSCIENCES LIMITED, Caulfield (AU)

(21) Appl. No.: 15/104,377

(22) PCT Filed: Dec. 9, 2014
(86) PCT No.: PCT/AU2014/050405

§ 371 Date: Jun. 14, 2016

(30) Foreign Application Priority Data
Dec. 16, 2013 (AU) 2013904912
Aug. 19, 2014 (AU) 2014903232

(51) Int. Cl. G01N 33/68

(52) U.S. Cl. CPC G01N 33/6893; G01N 2800/00; G01N 2800/56

(57) ABSTRACT

The present invention relates generally to a method of diagnosing and/or monitoring the development or progress of chronic fatigue syndrome. More particularly, the present invention relates to a method of diagnosing and/or monitoring the development or progress of chronic fatigue syndrome by analysis of activin β_{B} expression levels in a subject mammal or in a biological sample derived from said mammal. This may be achieved by screening for activin β_{B} in either monomeric form or in dimeric form. Still further, the ratio of the dimeric form of activin β_{B} relative to follistatin and activin A levels also provides a useful diagnostic indicator. In a related aspect there is provided a method for the treatment of chronic fatigue syndrome by downregulating the functional level of activin B.

### FIELD OF THE INVENTION

The present invention relates generally to a method of diagnosing and/or monitoring the development or progress of chronic fatigue syndrome. More particularly, the present invention relates to a method of diagnosing and/or monitoring the development or progress of chronic fatigue syndrome by analysis of activin β_{B} expression levels in a subject mammal or in a biological sample derived from said mammal. This may be achieved by screening for activin β_{B} in either monomeric form or in dimeric form. Still further, the ratio of the dimeric form of activin β_{B} relative to follistatin and activin A levels also provides a useful diagnostic indicator. In a related aspect there is provided a method for the treatment of chronic fatigue syndrome by downregulating the functional level of activin B.

### BACKGROUND OF THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Chronic fatigue syndrome is the common name for a group of significantly debilitating medical conditions characterised by persistent fatigue and other specific symptoms that last for a minimum of six months in adults (and three months in children or adolescents). The fatigue is not significantly relieved by rest, and is not caused by other medical conditions.

Symptoms of chronic fatigue syndrome include malaise after exertion; unrefreshing sleep, widespread muscle and joint pain, sore throat, headaches of a type not previously experienced, cognitive difficulties, chronic and severe mental and physical exhaustion, and other characteristic symptoms in a previously healthy and active person. Additional symptoms may be reported, including muscle weakness, increased sensitivity to light, sounds and smells, orthostatic intolerance, digestive disturbances, depression, painful and often slightly swollen lymph nodes, cardiac and respiratory problems. It is unclear if these symptoms represent co-morbid conditions or if they are produced by an underlying etiology of chronic fatigue syndrome. Chronic fatigue syndrome symptoms vary in number, type, and severity from person to person. Quality of life of persons with chronic fatigue syndrome can be extremely compromised.

Fatigue is a common symptom in many illnesses, but chronic fatigue syndrome is comparatively rare. Estimates of prevalence vary from 7 to 3,000 cases of chronic fatigue syndrome for every 100,000 adults; national health organizations have estimated more than one million Americans and approximately a quarter of a million people in the UK have chronic fatigue syndrome. Chronic fatigue syndrome occurs more often in women than men, and is less prevalent among children and adolescents.

The main feature of chronic fatigue syndrome is a type of exhaustion known as post-exertional malaise, ‘crash’ or ‘payback’. Research shows that people with chronic fatigue syndrome have a different physiological response to activity or exercise from other people. This includes abnormal exhaustion after any form of exertion, and a worsening of other symptoms. The response may be delayed, perhaps after 24 hours. Depending on the amount and type of exertion, it may result in post-exertional malaise for a few days, or serious relapses lasting weeks, months or even years.

People with chronic fatigue syndrome find that activities they once took for granted take an enormous toll on their health. For example, a short stroll, coffee with a friend, getting their child ready for school or catching the train to work, which caused no fatigue before, is followed by unusual tiredness that hikes longer than usual to go away.

Because chronic fatigue syndrome is a very complex, multi-system, chronic illness, many other symptoms will occur and must be present for diagnosis. These include:
neuro-cognitive problems (new difficulties in thinking, concentrating, memory loss, vision, clumsiness, muscle twitching or tingling)
disrupted sleep
pain or aches in the muscles, joints or head
a drop in blood pressure, feeling dizzy or pale
palpitations, increased heart rate or shortness of breath with exertion or on standing
allergies or sensitivities to light, smells, touch, sound, foods, chemicals and medications
gastrointestinal changes such as nausea, bloating, constipation, diarrhoea urinary problems
sore throat, tender lymph nodes and a flu-like feeling
marked weight change—extreme loss or gain
inability to cope with temperature changes.

Although there is agreement that chronic fatigue syndrome poses genuine threats to health, happiness and productivity, various physicians groups, researchers and patient advocates promote differing nomenclatures, diagnostic criteria, etiological hypotheses and treatments, resulting in controversy about many aspects of the disorder.

Accordingly, in the absence of a definitive etiology, the conclusive diagnosis of chronic fatigue syndrome is difficult and slow, due largely to the wide range of non-specific symptoms which characterise this disease. This fact in itself carries significant stress to individuals suffering from chronic fatigue syndrome, since doctors make a diagnosis by excluding all other illnesses after a person has had symptoms continually for six months. The person's results from routine medical tests will often be normal, but additional tests will show abnormalities. Similarly, the treatment of chronic fatigue syndrome is non-specific and of moderate effectiveness. Generally patients are treated with one or more of psychological and physical therapy as well as energy management strategies. Other treatments of chronic fatigue syndrome have been proposed but their effectiveness has not been confirmed. Medications thought to have promise in alleviating symptoms include antidepressant and immunomodulatory agents. The evidence for antidepressants is mixed, and their use remains controversial. Many chronic fatigue syndrome patients are sensitive to medications, particularly sedatives, and some patients report chemical and food sensitivities. Chronic fatigue syndrome patients have a low placebo response, especially to psychological-psychiatric interventions, perhaps due to patient expectations.

Accordingly, there is a significant need for the development of both a single, accurate diagnostic test and, further, more effective treatment regimes. In work leading up to the present invention, it has been determined that activin β_{B} (also known as inhibin β_{B}) levels are increased in patients who have developed chronic fatigue syndrome. The same is not true for activin β_{A} (also known as inhibin β_{A}). Accordingly, this provides a reliable and specific means for more quickly diagnosing chronic fatigue syndrome, particularly in patients exhibiting generalised symptoms which could be characteristic of several conditions. To this end, one may conveniently screen for the level of activin β_{B} in its monomeric form or its dimeric form (activin B) or in the context of a ratio with activin A or follistatin. Still further, the level of activin β_{B} increase is also indicative of the severity of the condition, with increasing severity of chronic fatigue syndrome being characterised by progressively increasing activin β_{B}.

In a related aspect, it has also been determined that reducing activin B levels in chronic fatigue syndrome patients provides an alternative treatment regime. Accordingly, this now provides a means of more effectively managing patients exhibiting symptoms of chronic fatigue syndrome, both in terms of providing a more accurate tool for diagnosing and/or monitoring chronic fatigue syndrome and an additional tool for use or instead of or together with existing diagnostic methods. Still further, an additional chronic fatigue syndrome treatment regime has been developed, for use either alone or together with one or more existing treatment regimes.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention is directed to a method for detecting chronic fatigue syndrome in a mammal, said method comprising screening for the level of activin β_{B} protein and/or gene expression in said mammal or in a biological sample derived from said mammal wherein an increase in the level of said protein and/or gene expression relative to normal levels is indicative of chronic fatigue syndrome.

In another aspect there is provided a method for detecting chronic fatigue syndrome in a mammal, said method comprising screening for the level of activin B protein and/or gene expression in said mammal or in a biological sample derived from said mammal wherein an increase in the level of said protein and/or gene expression relative to normal levels is indicative of chronic fatigue syndrome.

In a further aspect there is provided a method of detecting chronic fatigue syndrome in a mammal, said method comprising screening for the level of one or both of:
(i) activin B:follistatin protein and/or gene expression ratio;
(ii) activin B:activin A protein and/or gene expression ratio;
(iii) activin β_{B}:follistatin protein and/or gene expression ratio;
(iv) activin β_{B}:activin A protein and/or gene expression ratio; or
(v) activin β_{B}:activin β_{A} protein and/or gene expression ratio;
   in said mammal or in a biological sample derived from said mammal wherein an increase in the level of said ratio relative to normal levels is indicative of chronic fatigue syndrome.

In another further aspect, there is provided a method of detecting chronic fatigue syndrome in a mammal, which mammal is exhibiting one or more symptoms of:
post-exertional malaise
neuro-cognitive problems (new difficulties in thinking, concentrating, memory loss, vision, clumsiness, muscle twitching or tingling)
disrupted sleep
pain or aches in the muscles, joints or head
a drop in blood pressure, feeling dizzy or pale
palpitations, increased heart rate or shortness of breath with exertion or on standing
allergies or sensitivities to light, smells, touch sound, foods, chemicals and medications
gastrointestinal changes such as nausea, bloating, constipation, diarrhoea
urinary problems
sore throat, tender lymph nodes and a flu-like feeling
marked weight change—extreme loss or gain
inability to cope with temperature changes.
brain fog
difficulty maintaining an upright position, dizziness, balance problems or fainting
allergies or sensitivities to foods, odors, chemicals, medications, or noise
irritable bowel syndrome-like symptoms such as bloating, stomach pain, constipation, diarrhoea and nausea
chills and night sweats
visual disturbances (sensitivity to light, blurring, eye pain)
depression or mood problems (irritability, mood swings, anxiety, panic attacks)
   said method comprising screening for the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(v) activin β_{B}:activin β_{A} protein and/or gene expression ratio;
   in said mammal or in a biological sample derived from said mammal wherein an increase in the level or ratio of said protein and/or gene expression relative to normal levels is indicative of the onset of chronic fatigue syndrome.

Still another aspect of the present invention relates to a method for monitoring the progression of chronic fatigue syndrome in a maim at said method comprising screening for modulation of the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio,
(vi) activin β_{B}:activin A protein and/or gene expression ratio; or
(v) activin β_{B}:activin β_{A} protein and/or gene expression ratio;
   in said mammal.

Yet another aspect of the present invention provides a method for monitoring the progression of chronic fatigue syndrome in a mammal, said method comprising screening for modulation of the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}: activin A protein and/or gene expression ratio; or
(vii) activin β_{B}:activin β_{A} protein and/or gene expression ratio;
   in said mammal or in a biological sample derived from said mammal wherein an increase in the level, or ratio of said protein and/or gene expression relative to a previously obtained level is indicative of the worsening of said condition, a decrease in said level is indicative of an improvement in said condition and no change to said level is indicative of no significant change to the severity of said condition.

In yet a another further aspect there is provided a method of assessing the severity of chronic fatigue syndrome in a mammal, said method comprising determining the level of one or more of:
(i) activin β_{B} and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(vii) activin β_{B}:activin β_{A} protein and/or gene expression ratio;
   in said mammal or in a biological sample derived from said mammal wherein the higher the level or ratio of said protein and/or gene expression then the more severe the chronic fatigue syndrome.

In accordance with these aspects, in one particular embodiment said method comprises screening for the modulation of the level of one or more of:
(i) activin B:follistatin protein and/or gene expression ratio; or
(ii) activin B:activin A protein and/or gene, expression ratio.

In a related aspect there is provided a method of treating chronic fatigue syndrome in a mammal, said method comprising downregulating the functional level of activin B in said mammal.

In yet a further aspect there is provided a method of treating chronic fatigue syndrome in a mammal, which mammal is exhibiting one or more symptoms of:
post-exertional malaise
neuro-cognitive problems (new difficulties in thinking, concentrating, memory loss, vision, clumsiness, muscle twitching or tingling)
disrupted sleep
pain or aches in the muscles, joints or head
a drop in blood pressure, feeling dizzy or pale
palpitations, increased heart rate or shortness of breath with exertion or on standing allergies or sensitivities to tight, smells, touch, sound, foods, chemicals and medications
gastrointestinal changes such as nausea, bloating, constipation, diarrhoea
urinary problems
sore throat, tender lymph nodes and a flu-like feeling
marked weight change—extreme loss or gain
inability to cope with temperature changes.
brain fog (feeling like you're in a mental fog)
difficulty maintaining an upright position, dizziness, balance problems or fainting
allergies or sensitivities to foods, odors, chemicals, medications, or noise
irritable bowel syndrome-like symptoms such as bloating, stomach pain, constipation, diarrhoea and nausea
chills and night sweats
visual disturbances (sensitivity to light, blurring, eye pain)
depression or mood problems (irritability, mood swings, anxiety, panic attacks)
   said method comprising downregulating the functional level of activin B in said mammal.

### BRIEF DESCRIPTION OF THE DRAWINGS

FIG. 1 is a graphical representation depicting that Activin B, but not activin A, levels are elevated in patients diagnosed with chronic fatigue syndrome (CFS). Plasma concentrations of activin A (Act A, A) and activin B (Act B, B) in patients diagnosed with chronic fatigue syndrome (CFS) compared to control group (NR group). The data is also represented according to patient sex, with activin A and activin B levels in females shown in C and D and males shown in E and F. Data are presented as mean±SEM.

FIG. 2 is a graphical representation depicting that Follistatin levels are reduced in patients diagnosed with chronic fatigue syndrome (CFS). (A) Plasma concentrations of follistatin in patients diagnosed with chronic fatigue syndrome (CFS) compared to controls (NR group). Plasma follistatin concentrations in female (B) and male (C) CFS patients compared to same sex controls. Data are presented as mean±SEM.

FIG. 3 is a graphical representation depicting that activin to follistatin ratios and the ratio of activin B to activin A are elevated in patients diagnosed with chronic fatigue syndrome (CFS). Activin A:follistatin (ActA:Fst, A); activin B:follistatin (ActB:Fst, B) ratios and activin B:activin A (ActB/ActA) ratios in patients diagnosed with chronic fatigue syndrome (CFS) compared to controls (NR group). Data are presented as mean±SEM.

FIG. 4 is a graphical representation depicting Activin B levels relative to chronic fatigue severity (b). The figure also shows that the weighted standing time (WST) gives a good measure of CFS severity in patients and was used to categorize CFS patients into 3 groups depending on their fatigue status (a). Category 0: least severe (n=2) +healthy controls (n=17), all stood 20 mins at difficulty 0-2. Category 1: moderate severity (n=30); all stood 20 mins at difficulty 3-9. Group 2: most severe (n=15); all stood for <20 mins at difficulty 10-14. Data are presented as mean±SEM.

### DETAILED DESCRIPTION OF THE INVENTION

The present invention is predicated, in part, on the determination that an increased level of activin β_{B} is an accurate and highly sensitive indicator of the existence and severity of chronic fatigue syndrome. Most surprisingly, it has been found that levels of activin β_{B} are increased in chronic fatigue syndrome patients while activin A levels and levels of several inflammatory markers remain unchanged. Accordingly, the present invention provides a means of sensitively and accurately assessing chronic fatigue syndrome based on relative levels of activin β_{B}. Activin β_{B} levels can be assessed in terms of activin β_{B} in either its monomeric form or its dimeric form, such as in the context of the activin B homodimer. Still further, since activin β_{B} levels are selectively increased, in particular relative to activin A, one may also screen for changes to activin β_{B} related ratios, for example activin B:follistatin ratios and activin B:activin A ratios. This finding has therefore facilitated the development of a highly sensitive and informative assay directed to diagnosing, monitoring and determining the severity of chronic fatigue syndrome. In a related aspect, it has also been determined that reducing the functional level of activin B in chronic fatigue syndrome patients provides an additional treatment regime for use either alone or in conjunction with other treatment modalities.

Accordingly, one aspect of the present invention is directed to a method for detecting chronic fatigue syndrome in a mammal, said method comprising screening for the level of activin β_{B} protein and/or gene expression in said mammal or in a biological sample derived from said mammal wherein an increase in the level of said protein and/or gene expression relative to normal levels is indicative of chronic fatigue syndrome.

Reference to “activin β_{B}” should be understood as a reference to all forms of activin β_{B} and to fragments, derivatives, mutants or variants thereof. “Activin β_{B}” is also interchangeably referred to as “activin β_{B} subunit”. It should be understood to include reference to any isoforms which may arise from alternative splicing of activin β_{B} mRNA or mutant or polymorphic forms of activin β_{B}. Reference to “activin β_{B}” is not intended to be limiting and should be read as including reference to all forms of activin β_{B} including any protein encoded by the activin β_{B} subunit gene, any subunit polypeptide such as precursor forms which may be generated. Without limiting the present invention to any one theory CT mode of action, the activin β_{B} monomer will associate with other activin-related monomers to form a dimer. For example, known dimeric forms of activin β_{B} include the homodimeric activin B (β_{B}-β_{B}) and the heterodimeric AB (β_{A}-β_{B}), activin BC (β_{B}-β_{C}), activin BD (β_{B}-β_{D}) or activin BE (β_{B}-β_{E}) proteins.

In one embodiment, said activin β_{B} is screened for in monomeric form.

In another embodiment, said activin β_{B} is screened for in homodimeric form, that is as activin B.

According to this embodiment there is provided a method for detecting chronic fatigue syndrome in a mammal, said method comprising screening for the level of activin B protein and/or gene expression in said mammal or in a biological sample derived from said mammal wherein an increase in the level of said protein and/or gene expression relative to normal levels is indicative of chronic fatigue syndrome.

As detailed hereinbefore, in addition to measuring levels of the activin β_{B} monomer or homodimer, one may also screen for changes to activin B or activin β_{B} ratios relative to activin A, levels of which remain essentially unchanged, and follistatin.

Accordingly, in a further embodiment there is provided a method of detecting chronic fatigue syndrome in a mammal, said method comprising screening for the level of one or both of:
(i) activin B:follistatin protein and/or gene expression ratio;
(ii) activin B:activin A protein and/or gene expression ratio;
(iii) activin β_{B}:follistatin protein and/or gene expression ratio;
(iv) activin β_{B}:activin A protein and/or gene expression ratio; or
(v) activin β_{B}:activin β_{A} protein and/or gene expression ratio;
   in said mammal or in a biological sample derived from said mammal wherein an increase in the level of said ratio relative to normal levels is indicative of chronic fatigue syndrome.

Without limiting the present invention to any one theory or mode of action, chronic fatigue syndrome (CFS) is the common name for a medical condition characterised by persistent fatigue and other specific symptoms that last for a minimum of six months in adults (and three months in children or adolescents). The fatigue is not significantly relieved by rest, and is not caused by other medical conditions. Symptoms of chronic fatigue syndrome include, but are not limited to:
neuro-cognitive problems (new difficulties in thinking, concentrating, memory loss, vision, clumsiness, muscle twitching or tingling)
disrupted sleep
pain or aches in the muscles, joints or head
a drop in blood pressure, feeling dizzy or pale
palpitations, increased heart rate or shortness of breath with exertion or on standing
allergies or sensitivities to light, smells, touch, sound, foods, chemicals and medications
gastrointestinal changes such as nausea, bloating, constipation, diarrhoea
urinary problems
sore throat, tender lymph nodes and a flu-like feeling
marked weight change—extreme loss or gain
inability to cope with temperature changes.

Other common symptoms include:
brain fog (feeling like one is in a mental fog)
difficulty maintaining an upright position, dizziness, balance problems or fainting
allergies or sensitivities to foods, odors, chemicals, medications, or noise
irritable bowel syndrome-like symptoms such as bloating, stomach pain, constipation, diarrhoea and nausea
chills and night sweats
visual disturbances (sensitivity to light, blurring, eye pain)
depression or mood problems (irritability, mood swings, anxiety, panic attacks).

Reference to “chronic fatigue syndrome” should therefore be understood as a reference to a disease condition characterised by post-exertional malaise. This condition may also manifest with one or more of the additional symptoms detailed above. It should be understood, however, that these symptoms may fluctuate over short periods of time, even from hour to hour. Still further, patients may exhibit some but not all of these symptoms. Accordingly, the symptoms exhibited by a group of patients can be quite variable from one patient to the next. In one particular embodiment, said chronic fatigue syndrome is characterised by the following three criteria:
1. A new onset of severe fatigue for six consecutive months or greater duration which is unrelated to exertion, is not substantially relieved by rest, and is not a result of other medical conditions.
2. The fatigue causes a significant reduction of previous activity levels.
3. Four or more of the following symptoms that last six months or longer:
   impaired memory or concentration
   post-exertional malaise, where physical or mental exertions bring on “extreme, prolonged exhaustion and sickness”
   unrefreshing sleep
   muscle pain (myalgia)
   pain in multiple joints (arthralgia)
   headaches of a new kind or greater severity
   sore throat, frequent or recurring
   tender lymph nodes (cervical or axillary)

In another aspect, there is provided a method of detecting chronic fatigue syndrome in a mammal, which mammal is exhibiting one or more symptoms of:
post-exertional malaise
neuro-cognitive problems (new difficulties in thinking, concentrating, memory loss, vision, clumsiness, muscle twitching or tingling)
disrupted sleep
pain or aches in the muscles, joints or head
a drop in blood pressure, feeling dizzy or pale
palpitations, increased heart rate or shortness of breath with exertion or on standing
allergies or sensitivities to light, smells, touch, sound, foods, chemicals and medications
gastrointestinal changes such as nausea, bloating, constipation, diarrhoea
urinary problems
sore throat, tender lymph nodes and a flu-like feeling
marked weight change—extreme loss or gain
inability to cope with temperature changes.
brain fog
difficulty maintaining an upright position, dizziness, balance problems or fainting
allergies or sensitivities to foods, odors, chemicals, medications, or noise
irritable bowel syndrome-like symptoms such as bloating, stomach pain, constipation, diarrhoea and nausea
chills and night sweats
visual disturbances (sensitivity to light, blurring, eye pain)
depression or mood problems (irritability, mood swings, anxiety, panic attacks)
   said method comprising screening for the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(vii) activin β_{B}:activin β_{A} protein and/or gene expression ratio;
   in said mammal or in a biological sample derived from said mammal wherein an increase in the level or ratio of said protein and/or gene expression relative to normal levels is indicative of the onset of chronic fatigue syndrome.

Still without limiting the present invention in any way, although chronic fatigue syndrome is the most commonly used designator, this disease condition is also known by other names including, but not limited to, Akureyri disease, benign myalgic encephalomyelitis, chronic fatigue immune dysfunction syndrome, chronic infectious mononucleosis, epidemic myalgic encephalomyelitis, epidemic neuromyasthenia, Iceland disease, myalgic encephalomyelitis, myalgic encephalitis, myalgic encephalopathy, post-viral fatigue syndrome, raphe nucleus encephalopathy, Royal Free disease, fibromyalgia and Tapanui flu.

The term “mammal” as used herein includes humans, primates, livestock animals (eg. horses, cattle, sheep, pigs, donkeys), laboratory test animals (eg. mice, rats, guinea pigs), companion animals (eg. dogs, cats) and captive wild animals (eg. kangaroos, deer, foxes). Preferably, the mammal is a human or a laboratory test animal. Even more preferably, the mammal is a human.

The method of the present invention is predicated on the correlation of activin β_{B} monomeric or dimeric levels in patients with the normal levels of this molecule. The “normal level” is the level of activin β_{B} in a corresponding biological sample of a subject who has not developed chronic fatigue syndrome. Without limiting the present invention in any way, it is generally believed that the systemic level of activin B, to the extent that one is screening for this homodimer at the systemic level, in a normal individual is low (Ludlow et al., 2009).

In a related aspect, since it has been determined that activin B is selectively increased in chronic fatigue patients, but not activin A, the ratio of activin B:activin A is increased. Still further it has also been determined that in addition to the level of activin B being selectively increased in patients ho have developed chronic fatigue syndrome, the level of follistatin in these patients is decreased. Accordingly, the ratio of activin B:follistatin levels is significantly increased in these patients. Without limiting the present invention to any one theory or mode of action, these ratios can be calculated either using mRNA levels or using protein levels. To this end, since activin B levels are increased in patients with chronic fatigue syndrome and follistatin levels are decreased, the ratio figure of activin B to follistatin will be particularly significantly increased.

The normal level may be determined using a biological sample corresponding to the sample being analysed but which has been isolated from an individual who has not developed chronic fatigue syndrome. However, it would be appreciated that it is likely to be most convenient to analyse the test results relative to a standard result which reflects individual or collective results obtained from healthy individuals. This latter form of analysis is in fact the preferred method of analysis since it enables the design of kits which require the collection and analysis of a single biological sample, being a test sample of interest. The standard results which provide the normal level may be calculated by any suitable means which would be well known to the person of skill in the art. For example, a population of normal biological samples can be assessed in terms of the level of activin β_{B} (whether in monomeric or dimeric form) thereby providing a standard value or range of values against which all future test samples are analysed. It should also be understood that the normal level may be determined from the subjects of a specific cohort and for use with respect to test samples derived from that cohort. Accordingly, there may be determined a number of standard values or ranges which correspond to cohorts which differ in respect of characteristics such as age, gender, ethnicity or health status. Said “normal level” may be a discrete level, or a range of levels.

The term “modulation” refers to increases and decreases in activin β_{B} monomer or dirtier levels relative either to a normal reference level (or normal reference level range) or to an earlier activin β_{B} level result determined from the subject. A normal reference level is the activin β_{B} monomer or dimer level from a relevant biological sample of a subject or group of subjects which are not experiencing chronic fatigue syndrome. In a preferred embodiment, said normal reference level is the level determined from one or more subjects of a relevant cohort to that of the subject being screened by the method of the invention. By “relevant cohort” is meant a cohort characterised by one or more features which are also characteristic of the subject who is the subject of screening. These features include, but are not limited to age, gender, ethnicity or health status, for example.

Although the preferred method is to detect an increase in activin β_{B} monomer or dimer levels or ratios, as hereinbefore described, in order to diagnose chronic fatigue syndrome, the detection of a decrease in these levels may be desired under certain circumstances. For example, to monitor for improvement in the status of a chronic fatigue syndrome patient during the course of therapeutic treatment, thereby also enabling a clinician to assess the efficacy of the treatment regime. This aspect of the present invention also enables one to monitor the progression of chronic fatigue syndrome. By “progression” is meant the ongoing nature of chronic fatigue syndrome, such as its improvement, maintenance, worsening or a change in the level of its severity. To this end, it has also been determined that the level of activin β_{B} progressively increases as the severity of chronic fatigue syndrome increases. Accordingly, this enables an assessment being made in relation to the severity of the chronic fatigue syndrome being experienced by a patient at any given point in time, such as at diagnosis or during treatment.

Accordingly, another aspect of the present invention relates to a method for monitoring the progression of chronic fatigue syndrome in a mammal, said method comprising screening for modulation of the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(vii) activin β_{B}:activin β_{A} protein and/or gene expression ratio;
   in said mammal.

It should be understood that in accordance with this aspect of the present invention, activin β_{B} monomer or dimer levels or ratios will likely be assessed relative to one or more previously obtained levels from the patient being monitored. Where the level of activin β_{B} or ratio is reduced relative to an earlier obtained level, the condition of the mammal is improving. However, where the level or ratio of activin β_{B} is the same or higher, the chronic fatigue syndrome patient's condition is not improving.

Accordingly, one embodiment of the present invention therefore provides a method for monitoring the progression of chronic fatigue syndrome in a mammal, said method comprising screening for modulation of the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(vii) activin β_{B}:activin β_{A} protein and/or gene expression ratio;
   in said mammal or in a biological sample derived from said mammal wherein an increase in the level or ratio of said protein and/or gene expression relative to a previously obtained level is indicative of the or worsening of said condition, a decrease in said level is indicative of an improvement in said condition and no change to said level is indicative of no significant change to the severity of said condition.

In yet another aspect there is provided a method of assessing the severity of chronic fatigue syndrome in a mammal, said method comprising determining the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(vii) activin β_{B}:activin β_{A} protein and/or gene expression ratio;
   in said mammal or in a biological sample derived from said mammal wherein the higher the level or ratio of said protein and/or gene expression then the more severe the chronic fatigue syndrome.

Without limiting the present invention to any one theory or mode of action, changes in the severity of chronic fatigue syndrome in a patient can conveniently be determined by comparing an activin β_{B} monomer or dimer level or ratio measurement relative either to earlier obtained results for that patient or to a range of standard values.

In another embodiment, the mammal which is the subject of analysis is exhibiting one or more symptoms of:
post-exertional malaise
neuro-cognitive problems (new difficulties in thinking, concentrating, memory loss, vision, clumsiness, muscle twitching or tingling)
disrupted sleep
pain or aches in the muscles, joints or head
a drop in blood pressure, feeling dizzy or pale
palpitations, increased heart rate or shortness of breath with exertion or on standing
allergies or sensitivities to light, smells, touch, sound, foods, chemicals and medications
gastrointestinal changes such as nausea, bloating, constipation, diarrhoea
urinary problems
sore throat, tender lymph nodes and a flu-like feeling
marked weight change—extreme loss or gain
inability to cope with temperature changes.
brain fog
difficulty maintaining an upright position, dizziness, balance problems or fainting
allergies or sensitivities to foods, odors, chemicals, medications, or noise
irritable bowel syndrome-like symptoms such as bloating, stomach pain, constipation, diarrhoea and nausea
chills and night sweats
visual disturbances (sensitivity to light, blurring, eye pain)
depression or mood problems (irritability, mood swings, anxiety, panic attacks).

Reference to a “biological sample” should be understood as a reference to an sample of biological material derived from a mammal such as, but not limited to cellular material, tissue biopsy specimens or bodily fluid (e.g. cerebrospinal fluid, (e.g. whole blood, plasma or serum)) or urine. The biological sample which is tested according to the method of the present invention may be tested directly or may require some form of treatment prior to testing. For example, the separation of serum or plasma from a whole blood sample before analysis. To the extent that the biological sample is not in liquid form (e.g. buccal swab), (if such form is required for testing) it may require the addition of a reagent, such as a buffer, to mobilise the sample.

The biological sample may be directly tested or else all or some of the nucleic acid material or protein present in the biological sample may be isolated prior to testing. In yet another example, the sample may be partially purified or otherwise enriched prior to analysis. For example, to the extend that a biological sample comprises a very diverse cell population, it may be desirable to select out a sub-population of particular interest if mRNA is the subject of analysis. It is within the scope of the present invention for the target nucleic acid or protein molecule to be pre-treated prior to testing, for example inactivation of live virus or being run on a gel. It should also be understood that the biological sample may be freshly harvested or it may have been stored (for example by freezing) prior to testing or otherwise treated prior to testing (such as by undergoing culturing).

The choice of what type of sample is most suitable for testing in accordance with the method disclosed herein will be dependent on the nature of the situation.

As detailed hereinbefore reference to “expression” should be understood as a reference to the transcription and/or translation of a nucleic acid molecule. Reference to “RNA” should be understood to encompass reference to any form of RNA, such as primary RNA or mRNA. Without limiting the present invention in any way, the modulation of gene transcription leading to increased or decreased RNA synthesis will also correlate with the translation of these RNA transcripts (such as mRNA) to a protein product. Accordingly, the present invention also extends to detection methodology which is directed to screening for modulated levels or patterns of activin β_{B} as an indicator of chronic fatigue syndrome. Although one method is to screen for mRNA transcripts and/or the corresponding protein product, it should be understood that the present invention is not limited in this regard and extends to screening for any other form of expression product such as, for example, a primary RNA transcript.

The term “protein” should be understood to encompass peptides, polypeptides and proteins (including protein fragments). The protein tray be glycosylated or unglycosylated and/or may contain a range of other molecules fused, linked, bound or otherwise associated to the protein such as amino acids, lipids, carbohydrates or other peptides, polypeptides or proteins. Reference herein to a “protein” includes a protein comprising a sequence of amino acids as well as a protein associated with other molecules such as amino acids, lipids, carbohydrates or other peptides, polypeptides or proteins.

Reference to a “fragment” should be understood as a reference to a portion of the subject nucleic acid molecule or protein. This is particularly relevant with respect to screening for modulated RNA levels since these are inherently unstable molecules and may be screened for in samples which express high levels of enzymes. In this case the subject RNA is likely to have been degraded or otherwise fragmented. One may therefore actually be detecting fragments of the subject RNA molecule, which fragments are identified by virtue of the use of a suitably specific probe.

Methods of screening for levels or ratios of activin β_{B} monomer or dimer, activin β_{A} monomer or dimer or follistatin can be achieved by any suitable method which would he well known to persons of skill in the art. In this regard, it should be understood that reference to screening for the level of protein and/or gene expression “in a mammal” is intended as a reference to the use of any suitable technique which will provide information in relation to the level of expression of activin β_{B} monomer or dinner in the relevant tissue of the mammal. These screening techniques include both in vivo screening techniques, as hereinafter described, as well as the in vitro techniques which are applied to a biological sample extracted form said mammal. Such in vitro techniques are likely to be preferred due to their significantly more simplistic and routine nature.

Since the present invention is predicated on screening for changes to the level or ratios of activin β_{B} monomer or diner or activin B:follistatin ratios, such changes can in fact be screened fear at the protein level or at the nucleic acid level, such as by screening for increases in the level of the relevant mRNA transcripts. The person of skill in the art will determine the most appropriate means of analysis in any given situation. However it is generally preferred that screening be performed in the context of protein molecules due to the relative simplicity of the techniques which are likely to be utilised. Nevertheless in certain situations, and in the context of particular biological samples, it may be desirable or otherwise useful to directly analyse gene transcription.

As described above, means of screening for changes in levels or ratios of activin β_{B} monomer or diner (herein referred to as “the marker”) in an individual, or biological sample derived therefrom, can be achieved by any suitable method, which would be well known to the person of skill in the art, such as but not limited to:

(i) In vivo detection of the marker. Molecular Imaging may be used following administration of imaging probes or reagents capable of disclosing altered expression levels of the marker snRNA or protein expression product in tissues. Molecular imaging (Moore, A., Basilion, J., Chiocca, E., and Weissleder, R., BBA, 1402:239-249, 1988; Weissleder, R., Moore, A., Ph.D.,. Mahmood-Bhorade, U., Benveniste, H., Chiocca, E. A., Basilion, J. P. Nature Medicine, 6:351-355, 2000) is the in vivo imaging of molecular expression that correlates with the macro-features currently visualized using “classical” diagnostic imaging techniques such as X-Ray, computed tomography (CT), MRI, Positron Emission Tomography (PET) or endoscopy.
(ii) Detection of up-regulation of mRNA expression in the cells by Fluorescent In Situ Hybridization (FISH), or in extracts from the cells by technologies such as Quantitative Reverse Transcriptase Polymerase Chain Reaction (RT-qPCR) or Flow cytometric qualification of competitive RT-PCR products (Wedemeyer, N., Potter, T., Wetzlich, S. and Gohde, W. Clinical Chemistry 48:9 1398-1405, 2002), array technologies or non-PCR amplification techniques, including isothermal techniques.

For example, a labelled polynucleotide encoding the marker may be utilized as a probe in a Northern blot of an RNA extract. Preferably, a nucleic acid extract from the animal is utilized in concert with oligonucleotide primers corresponding to sense and antisense sequences of a polynucleotide encoding the marker, or flanking sequences thereof, in a nucleic acid amplification reaction such as RT PCR, real time PCR or SAGE. A variety of automated solid-phase detection techniques are also appropriate. For example, a very large scale immobilized primer arrays (VLSIPS™) are used for the detection of nucleic acids as, for example, described by Fodor et al., 1991 and Kazal et al., 1996. The above genetic techniques are well known to persons skilled in the art.

For example, to detect the marker encoding RNA transcripts, RNA is isolated from a cellular sample suspected of containing the marker RNA. RNA can be isolated by methods known in the art, e.g. using TRIZOL™ reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs arc then amplified with sequence-specific oligonucleotides in PCR reactions to yield an amplified product.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al., 1987; Erlich, 1989.

To detect the amplified product, the reaction mixture may be subjected to agarose gel electrophoresis or other convenient separation technique and the relative presence of the marker specific amplified DNA detected. For example, the marker amplified DNA may be detected using Southern hybridization with a specific oligonucleotide probe or comparing is electrophoretic mobility with DNA standards of known molecular weight. Isolation, purification and characterization of the amplified marker DNA may be accomplished by excising or eluting the fragment from the gel (for example, see references Lawn et al., 1981; Goeddel et al., 1980), cloning the amplified product into a cloning site of a suitable vector, such as the pCRII vector (Invitrogen), sequencing the cloned insert and comparing the DNA sequence to the known sequence of the marker. The relative amounts of the marker mRNA and cDNA can then be determined. The amplified product may also be detected using SYBR green technology as for quantitative or real time PCR.

(iii) Measurement of marker protein levels in cell extracts or blood or other suitable biological sample, either qualitatively or quantitatively, for example by immunoassay, utilising immunointeractive molecules such as monoclonal antibodies.

In one example, one may seek to detect the marker-immunointeractive molecule complex formation. For example, an antibody or fragment having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to “Current Protocols in Immunology”, 1994 which discloses a variety of immunoassays which may be used in accordance with the present invention. Immunoassays may include competitive assays. It will be understood that the present invention encompasses qualitative and quantitative immunoassays.

Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen. The antigen in this case is the marker or a fragment thereof.

Assays which are designed to detect one or more different antigens (e.g. activin β_{B}, activin B, activin A and/or follistatin) arc particularly favoured for use in the present invention. A number of variations of these assays exist, all of which are intended to be encompassed by the present invention. Briefly, in a typical sandwich assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the sandwich assay include a simultaneous assay, in which both sample and labelled antibody are added simultaneously to the bound antibody in a competitive assay format. These techniques are well known to those skilled in the art, including minor variations as will be readily apparent.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody generally has a reporter molecule associated therewith that is used to indicate the binding of the second antibody to the antigen. The amount of labelled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like. A large number of enzymes suitable for use as reporter molecules is disclosed in U.S. Pat. No. 4,366,241, U.S. Pat. No. 4,843,000, and U.S. Pat. No. 4,849,338. Suitable enzymes useful in the present invention include alkaline phosphatase horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al., International Publication No. WO 93/06121. Reference also may be made to the fluorochromes described in U.S. Pat. No. 5,573,909 (Singer et al), U.S. Pat. No. 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second a antibody, giving a qualitative visual signal, which may be further quantitated, usually spectrophotometrically, to give an indication of the amount of antigen which was present in the sample.

Alternately, fluorescent compounds, such as fluorescein, rhodamine or lanthanide chelates, such as europium, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.

(iv) The use of aptamers in screening for nucleic acid molecules or protein expression products.
(v) Determining altered protein expression based on any suitable functional test, enzymatic test or immunological test in addition to those detailed above.

As detailed above, any suitable technique may be utilised to detect the markers or then encoding nucleic acid molecule. The nature of the technique which is selected for use will largely determine the type of biological sample which is required for analysis. Such determinations are well within the scope of the person of skill in the art. Typical samples which one may seek to analyse are blood samples.

In a related aspect, the present invention also provides a means for treating chronic fatigue syndrome in a mammal by down-regulating the functional level of activin B.

Accordingly, in a related aspect there is provided a method of treating chronic fatigue syndrome in a mammal, said method comprising downregulating the functional level of activin B in said mammal.

There is also provided an activin B antagonist for use in treating chronic fatigue syndrome in a mammal.

Reference to “chronic fatigue syndrome” and “activin B” should understood to have the same meaning as hereinbefore defined.

In one embodiment, there is provided a method of treating chronic fatigue syndrome in a mammal, which mammal is exhibiting one or more symptoms of:
post-exertional malaise
neuro-cognitive problems (new difficulties in thinking, concentrating, memory loss, vision, clumsiness, muscle twitching or tingling)
disrupted sleep
pain or aches in the muscles, joints or head
a drop in blood pressure, feeling dizzy or pale
palpitations, increased heart rate or shortness of breath with exertion or on standing
allergies or sensitivities to light, smells, touch, sound, foods, chemicals and medications
gastrointestinal changes such as nausea, bloating, constipation, diarrhoea
urinary problems
sore throat, tender lymph nodes and a flu-like feeling
marked weight change—extreme loss or gain
inability to cope with temperature changes.
brain fog (feeling like you're in a mental fog)
difficulty maintaining an upright position, dizziness, balance problems or fainting
allergies or sensitivities to foods, odors, chemicals, medications, or noise
irritable bowel syndrome-like symptoms such as bloating, stomach pain, constipation, diarrhoea and nausea
chills and night sweats
usual disturbances (sensitivity to light, blurring, eye pain)
depression or mood problems (irritability, mood swings, anxiety, panic attacks)
   said method comprising downregulating the functional level of activin B in said mammal.

In terms of downregulating the “functional level” of activin B, this should be understood to mean the level of activin B which is functional. It would be appreciated by the person of skill in the art that the functional level of activin B can be downregulated either by reducing absolute levels of activin β_{B} monomer and homodimer or by antagonising the functional activity of activin B such that its effectiveness is decreased. Even the partial antagonism of activin B may act to reduce, although not necessarily eliminate, the functional effectiveness of activin B.

In terms of achieving the downregulation of activin B, means for achieving this objective would be well known to the person of skill in the art and include, but are not limited to:
(i) Introducing into a cell a proteinaceous or non-proteinaceous molecule which downregulates the transcriptional and/or translational regulation of a gene, wherein this gene may be the activin β_{B} gene or functional portion thereof or some other gene or gene region (e.g. promoter region) which directly or indirectly modulates the expression of the activin β_{B} gene; or
(ii) Introducing a proteinaceous or non-proteinaceous molecule which functions as an antagonist to the activin B expression product.

The proteinaceous molecules described above may be derived from any suitable source such as natural, recombinant or synthetic sources and includes fusion proteins, variants or molecules which have been identified following, for example, natural product screening. In another example, one may utilize a genetically modified variant, such as a modified activin B molecule in which the prodomain has been modified to create an activin B antagonist. The reference to non-proteinaceous molecules may be, for example, a reference to a nucleic acid molecule or it may be a molecule derived front natural sources, such as for example natural product screening, or may be a chemically synthesised molecule. The present invention contemplates small molecules capable of acting as antagonists. Antagonists may be any compound capable of blocking, inhibiting or otherwise preventing activin B from carrying out its normal biological function. Antagonists include monoclonal antibodies and antisense nucleic acids which prevent transcription or translation of activin β_{B} genes or mRNA in mammalian cells. Modulation of expression may also be achieved utilising antigens, RNA, ribosomes, DNAzymes, aptamers, antibodies or molecules suitable for use in cosuppression. Suitable antisense oligonucleotide sequences (single stranded DNA fragments) of activin B may be created or identified by their ability to suppress the expression of activin β_{B}. The production of antisense oligonucleotides for a given protein is described in, for example, Stein and Cohen, 1988 (Cancer Res 48:2659-2668) and van der Krol et al., 1988 (Biotechniques 6:958-976). Antagonists also include any molecule that prevents activin B interacting with its receptor.

In the context of antibodies, the present invention envisages the use of any suitable form of antibody including catalytic antibodies or derivatives, homologues, analogues or mimetics of said antibodies. Such antibodies may be monoclonal or polyclonal and may be selected from naturally occurring activin B or its subunits or may be specifically raised to the activin B dimer or its monomers (herein referred to as the “antigen”). In the case of the latter, the antigen may first need to be associated with a carrier molecule. Alternatively, fragments of antibodies may be used such as Fab fragments or Fab′₂ fragments. Furthermore, the present invention extends to recombinant and synthetic antibodies and to antibody hybrids. A “synthetic antibody” is considered herein to include fragments and hybrids of antibodies. The antigen can also be used to screen for naturally occurring antibodies.

Both polyclonal and monoclonal antibodies are obtainable by immunization with the antigen or derivative, homologue, analogue, mutant, or mimetic thereof and either type is utilizable therapeutically. The methods of obtaining both types of sera are well known in the art. Polyclonal sera are less preferred but are relatively easily prepared by injection of a suitable laboratory animal with an effective amount of the antigen, or antigenic parts thereof, collecting serum from the animal, and isolating specific sera by any of the known immunoadsorbent techniques. Although antibodies produced by this method are utilizable, they are generally less favoured because of the potential heterogeneity of the product.

The use of monoclonal antibodies is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman 1981, Basic Facts about Hybridomas, in Compendium of Immunology Vol II, ed. by Schwartz; Kohler and Milstein 1975, Nature 256:495-499; Kohler and Milstein 1976, Eur J Immun 6:511-519).

Preferably, the antibody of the present invention specifically binds the antigen. By “specifically binds” is meant high avidity and/or high affinity binding of an antibody to a specific antigen. Antibody binding to its epitope on this specific antigen is stronger than binding of the same antibody to any other epitope, particularly those that may be present in molecules in association with, or in the same sample, as the specific antigen of interest. Antibodies that bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g. by use of appropriate controls.

The proteinaceous and non-proteinaceous molecules referred to, above, are herein collectively referred to as “modulatory agents”. To the extent that it is sought to decrease activin B activity, said modulatory agent is preferably:
(i) Follistatin. This may be administered either as a protein or its overexpression may be induced in vivo such as via the adenovirus mediated system described by Takabe et al. 2003 (Hepatology 38:1107-1115).
(ii) Any agent that upregulates the expression or functioning of the α subunit of inhibin. The α subunit can dimerise with the β subunits of activin B to form inhibin, thereby effectively downregulating activity B levels.
(iii) Inhibin. This molecule can bind to β-glycan and inhibit the actions of activin B via its receptor. See for example the mechanism described by Xu et al. 1995 (J Biol Chem 270:6308-6313) or the use of the Smad7 antagonist (Bernard et al. 2004, Molecule Endocrinol 18:606-623).
(iv) Activin B neutralising antibody. For example, as described in Poulaki et al. 2004 (Am J Pathol 164:1293-1302).
(v) Activin B mutants which inhibit native activin B from binding to its receptor. For example as described in Harrison et at 2004, (J. Biol Chem 279:28036-28044), or modifications of the prodomain of the activin B propeptide (see Makanji Y et al. 2011 Generation of a specific activin B antagonist by modification of the activin B propeptide. Endocrinol 152:3758-3768).
(vi) Transfection or treatment with a mutant activin B receptor which prevents normal activin B signalling or a soluble activin B receptor which acts as a competitive inhibitor. See for example, the system described by Maeshima et al. 2004 (Endocrinology 145:3739-3745).
(vii) An activin B antisense oligonucleotide.
(viii) A thrombin antagonist such as lepirudin.
(ix) The Cripto protein. This protein is required for nodal signaling. However, it specifically binds to activin B and inhibits it's signaling (Adkins et al. 2003).
(x) Any inhibitor of the ALK7 or ALK3 receptors through which activin B can signal.

In this regard, reference to “follistatin” should be read as including reference to all forms of follistatin including, by way of example, the three protein cores and six molecular weight forms which have been identified as arising from the alternatively spliced mRNAs FS315 and FS288. Accordingly, it should also be understood to include reference to any isoforms which may arise from alternative splicing of follistatin mRNA or mutant or polymorphic forms of follistatin. It should still further be understood to extend to any protein encoded by the follistatin gene, any subunit polypeptide, such as precursor forms which may be generated, and any follistatin protein, whether existing as a monomer, multimer or fusion protein. An analogous definition, applies to “inhibin”.

Other forms of follistatin which are suitable for use in the present invention include:
(i) Wild-type follistatin (FS), comprising an N-terminal domain (ND) followed by three follistatin domains (FSD1, FSD2 and FSD3) with a heparin-binding sequence located in FSD1 (amino acid sequence positions 72-86), and all known isoforms thereof.
(ii) Wild-type follistatin-like 3 protein (FSTL3), which is also known as follistatin-related gene product (FLRG) and follistatin-related protein (FSRP), comprising an N-terminal domain (N3D) followed by two follistatin-like 3 domains (PS3D1 and FS3D2), and all known isoforms thereof.
(iii) Follistatin analogue having the structure ND-FSD1-FSD2 (i.e. wild-type minus FSD3).
(iv) Analogues of (i) and (iii) above with FSD1 substituted by FSD1′, where FSD1 represents FSD1 with heparin-binding site removed.
(v) Analogues of (i) and (iii) above with FSD1 substituted by FSD1*, where FSD1 represents FSD1 with sequence prior to and including the heparin-binding sequence removed.
(vi) Hybrid forms of (i) and (iii) above where at least one of the domains is substituted by a corresponding FSTL3 domain N3D, FS3D1 and FS3D2.
(vii) Hybrid forms of (ii) above where at least one of the domains is substituted by a corresponding FS domain ND, FSD1, FSD1′, FSD1* and FSD2.
(viii) Any of the above proteins modified by one or more deletions, insertions and/or mutations in ND, N3D, FSD1, FSD1′, FSD1*, FS3D1, FSD2, FS3D2, and FSD3 provided the modified protein functions as an activin B antagonist.
(ix) Genetically modified forms of follistatin which have been modified to preferentially antagonize activin B over other activin or follistatin targets.
   (Jennifer N. Cash, Elizabeth B. Angerman, Henry T. Keutmann, and Thomas B. Thompson 2012 Characterization of Follistatin-Type Domains and Their Contribution to Myostatin and Activin A Antagonism. Mol Endocrinol, 26(7):1167-1178; Henry T. Keutmann, Alan L. Schneyer and Israel Sidis 2004 The Role of Follistatin Domains in Follistatin Biological Action. Mol Endocrinol, 18(1):228-240).

Screening for the modulatory agents hereinbefore defined can be achieved by any one of several suitable methods including, but in no way limited to, contacting a cell comprising the activin β_{B} gene or functional equivalent or derivative thereof with an agent and screening for the downregulation of activin B protein production or functional activity, downregulation of the expression of a nucleic acid molecule encoding activin β_{B} or downregulation of the activity or expression of a downstream activin B cellular target. Detecting such downregulation can be achieved utilising techniques such as Western blotting, electrophoretic mobility shift assays and/or the readout of reporters of activin B activity such as luciferases, CAT and the like.

It should be understood that the activin β_{B} gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which downregulate the functional level of activin B, at either the nucleic acid or expression product levels, or the gene may require activation—thereby providing a model useful for, inter alia, screening for agents which up-regulate activin β_{B} monomer or homodimer expression. Further, to the extent that an activin β_{B} nucleic acid molecule is transfected into a cell, that molecule may comprise the entire activin β_{B} gene or it may merely comprise a portion of the gene such as the portion which regulates expression of the activin B product. For example, the activin β_{B} promoter region may be transfected into the cell which is the subject of testing. In this regard, where only the promoter is utilised, detecting modulation of the activity of the promoter can he achieved, for example, by ligating the promoter to a reporter gene. For example, the promoter may be ligated to luciferase or a CAT reporter, the downregulation of expression of which gene can be detected via modulation of fluorescence intensity or CAT reporter activity, respectively. In another example, the subject of detection could be a downstream activin B regulatory target, rather than activin B itself. Yet another example includes activin B binding sites ligated to a minimal reporter.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the activin β_{B} nucleic acid molecule or expression product itself or which modulate the expression of an upstream molecule, which upstream molecule subsequently downregulates activin β_{B} monomer or homodimer expression or expression product activity. Accordingly, these methods provide a mechanism of detecting agents which either directly or indirectly modulate activin β_{B} monomer or homodimer expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention said agent is associated with a molecule which permits its targeting to a localised region.

The subject proteinaceous or non-proteinaceous molecule may act either directly or indirectly to downregulate the expression of activin β_{B} monomer or homodimer or the activity of the activin β_{B} monomer or homodimer expression product. Said molecule acts directly if it associates with the activin B nucleic acid molecule or expression product to modulate expression or activity, respectively. Said molecule acts indirectly if it associates with a molecule other than the activin β_{B} nucleic acid molecule or activin B expression product which other molecule either directly or indirectly downregulates the expression or activity of the activin β_{B} nucleic acid molecule or activin B expression product, respectively. Accordingly, the method of the present invention encompasses the regulation of activin β_{B} nucleic acid molecule expression or activin B expression product activity via the induction of a cascade of regulatory steps.

The term “expression” refers to the transcription and translation of a nucleic acid molecule. Reference to “expression product” is a reference to the product produced from the transcription and translation of a nucleic acid molecule.

A “variant” or “mutant” should be understood to mean molecules which exhibit at least some of the functional activity of the form of molecule (e.g. activin B or follistatin) of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of follistatin, for example, which exhibits similar and suitable functional characteristics to that of the follistatin which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening. Antagonistic agents can also be screened for utilising such methods.

For example, libraries containing small organic molecules may be screened, wherein organic molecules having a large number of specific parent group substitutions are used. A general synthetic scheme may follow published methods (e.g., Bunin et al. 1994, Proc Natl Acad Sci USA 91:4708-4712; DeWitt et al. 1993 Proc Natl Acad Sci USA 90:6909-69131. Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

The present invention is also directed to useful aptamers. In one embodiment, an aptamer is a compound that is selected in vitro to bind preferentially to another compound (in this case the identified proteins), in one aspect, aptamers are nucleic acids or peptides. Random sequences can be readily generated from nucleotides or amino acids (naturally occurring and/or synthetically made) in large numbers but of course they need not be limited to these. In another aspect, the nucleic acid aptamers are short strands of DNA that hind protein targets, such as oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. Transfer of large amounts of a chemically stabilized aptamer into cells can result in specific binding to a polypeptide of interest, thereby blocking its function. For example, see the following publications describing in vitro selection of aptamers: Klug et al., 1994, Mol Biol Rep 20:97-107; Wallis et al. 1995, Chem Biol 2:543-552; Ellington 1994, Curr Biol 4:427-429; Lato et al. 1995, Chem Biol 2:291-303; Conrad et al. 1995, Mol Divers 1:69-78; and Uphoff et al. 1996, Curr Opin Struct Biol 6:281-2871.

Certain RNA inhibiting agents may be utilized to inhibit the expression or translation of messenger RNA (“mRNA”) that is associated with a phenotype of interest. Examples of such agents suitable for use herein include, but are not limited to short interfering RNA (“siRNA”), ribozymes, aptamers, and antisense oligonucleotides.

In some instances, a range of 18-25 nucleotides is the most preferred size for siRNAs. siRNAs can also include short hairpin RNAs in which both strands of an siRNA duplex are included within a single RNA molecule. siRNA includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the RNA or internally (at one or more nucleotides of the RNA).

In one embodiment, the RNA molecules contain a 3′ hydroxyl group. Nucleotides in the RNA molecules of the present invention can also comprise non-standard nucleotides, including non-naturally occurring nucleotides or deoxyribonucleotides. Collectively, all such altered RNAs are referred to as analogues of RNA. siRNAs of the present invention need only be sufficiently similar to natural RNA that it has the ability to mediate RNA interference (RNAi).

Methods for designing double stranded RNA to inhibit gene expression in a target cell are known (see, e.g., U.S. Pat. No. 6,506,559; Elbashir et al. 2002, Methods 26:199-213; Chalk et al. 2004, Biochem Biophys Res Commun 319:264-274; Cui et al. 2004, Comput Methods Programs Biomed 75:67-73; Wang et al. 2004, Bioinformatics 20:1818-1820). For example, design of siRNAs (including hairpins) typically follow known thermodynamic rules (see, Schwarz, et al. 2003, Cell 115:199-208; Reynolds et al. 2004, Nat Biotechnol. 22:326-330; Khvorova et al. 2003, Cell 115:209-216). Many computer programs are available for selecting regions of a sequence that are suitable target sites. These include programs available through commercial sources such as Ambion, Dharmacon, Promega, Invitrogen, Ziagen, and GenScript as well as non-commercial sources such as EMBOSS, The Wistar Institute, Whitehead Institute, and others.

For example, design can be based on the following considerations. Typically, shorter sequences, less than about 30 nucleotides are selected. The coding region of the mRNA is usually targeted. The search for an appropriate target sequence optionally begins 50-100 nucleotides downstream of the start codon, as untranslated region binding proteins and/or translation initiation complexes may interfere with the binding of the siRNA endonuclease complex. Some algorithms, e.g., based on the work of Elbashir et al. 2000 (Methods 26:199-213) search for a selected sequence motif and select hits, with approximately 50% G/C-content (30% to 70% has also worked). If no suitable sequences are found, the search is extended.

Other nucleic acids, e.g., ribozymes, antisense, can also be designed based on known principles. For example, Sfold (see, e.g., Ding, et al., Nucl Acids Res 32 Web Server issue, W135-W141; Ding & Lawrence 2003, Nucl Acids Res 31; 7280-7301; and Ding & Lawrence 2001, Nucl Acids Res 20:1034-1046) provides programs relating to designing ribozymes and antisense, as well as siRNAs.

In one embodiment, downregulation of the functional level of activin B is achieved by administering follistatin, inhibin, an antibody directed to activin B, an activin β_{B} antisense oligonucleotide, a non-functional activin B molecule which competitively inhibits binding to the activin B receptor or a mutant or soluble activin B receptor which inhibits normal activin B signalling.

An “effective amount” means an amount necessary to at least partly attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. it is expected that the amount will fall in a relatively broad range that can be determined through routine trials.

Reference herein to “therapeutic” and “prophylactic” treatment is to be considered in its broadest context. The term “therapeutic” does not necessarily imply that the condition is treated until total recovery. Similarly, “prophylactic” does not necessarily mean that the patient will not develop some level of chronic fatigue syndrome. Accordingly, treatment includes amelioration of the symptoms of chronic fatigue syndrome.

The pharmaceutical compositions of the invention can be administered in a variety of unit dosage forms depending upon the method of administration. Dosages for typical modulatory pharmaceutical compositions are well known to those of skill in the art. Such dosages are typically advisory in nature and are adjusted depending on the particular therapeutic context, patient or organ tolerance, etc. The amount of agent adequate to accomplish this is defined as a “therapeutically effective dose.” The dosage schedule and amounts effective for this use, i.e., the “dosing regimen,” will depend upon a variety of factors, including the condition of the heart, the pre-existence or not of damage onset, the pharmaceutical formulation and concentration of active agent, and the like. In calculating the dosage regimen for an organ, the mode of administration also is taken into consideration. The dosage regimen must also take into consideration the pharmacokinetics, i.e., the pharmaceutical composition's rate of absorption, bioavailability, metabolism, clearance, and the like. (See, e.g., the latest Remington's; Egleton and Davis 1995 Peptides 18; 1431-1439; Langer 1990 Science 249:1527-1533).

The pharmaceutical composition which comprises the modulatory agents hereinbefore described may be administered by any convenient means and is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The composition may be administered in a convenient manner such as by the oral, intravenous (where water soluble), intraperitoneal, intramuscular, subcutaneous, intradermal or suppository routes or implanting (e.g. using slow release molecules). The antagonist may be administered as a nasal or oral spray or in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drop patch and implant.

In accordance with these methods, the composition defined in accordance with the present invention may be coadministered with one or more other compounds or molecules. By “coadministered” is meant simultaneous administration in the same formulation or in two different formulations via the same or different routes or sequential administration by the same or different routes. For example, the subject composition may be administered together with an agent in order to enhance its effects. By “sequential” administration is meant a time difference of from seconds, minutes, hours or days between the administration of the two types of molecules. These molecules may be administered in any order.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal antagonists, for example, parabens, chlorobutanol, phenol, sorbic acid, thimerosal and the like. In many cases, it will be preferable to include isotonic antagonists, for example, sugars or sodium chloride. Prolonged absorption of the injectable compositions can be brought about by the use in the compositions of antagonists delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum, drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets buccal tablets, troches, capsules, elixirs, suspensions, syrups, wafers, and the like. Such compositions and preparations should contain at least 1% by weight of active compound. The percentage of the compositions and preparations may of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating antagonist such as corn starch, potato starch, alginic acid and the like; a lubricant such as magnesium stearate; and a sweetening antagonist such as sucrose, lactose or saccharin may be added or a flavouring antagonist such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening antagonist, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding said antagonist or follistatin, such as antisense RNA, microRNA or peptide antagonist. The vector may, for example, be a viral vector.

Various methods of transferring or delivering DNA to cells for expression of the gene product protein, otherwise referred to as gene therapy, are disclosed in Gene Transfer into Mammalian Somatic Cells in vivo, N. Yang, Crit. Rev. Biotech. 12(4):335-356 (1992), which is hereby incorporated by reference.

Gene transfer methods for gene therapy fall into three broad categories: physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer.

Chemical methods of gene therapy may involve a lipid based compound, not necessarily a liposome, to ferry the DNA across the cell membrane. Lipofectins or cytofectins, lipid-based positive ions that bind to negatively charged DNA, may be used to cross the cell membrane and provide the DNA into the interior of the cell. Another chemical method may include receptor-based endocytosis, which involves binding a specific ligand to a cell surface receptor and enveloping and transporting it across the cell membrane.

Many gene therapy methodologies employ viral vectors such as retrovirus vectors to insert genes into cells. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors may be selected from the group including, but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors and are preferred. Adenoviral vectors may be delivered bound to an antibody that is in turn bound to collagen coated stents.

Mechanical methods of DNA delivery may be employed and include, but are not limited to, fusogenic lipid vesicles such as liposomes or other vesicles for membrane fusion, lipid particles of DNA incorporating cationic lipid such as lipolectin, polylysine-mediated transfer of DNA, direct injection of DNA, such as microinjection of DNA into germ or somatic cells, pneumatically delivered DNA-coated particles, such as the gold particles used in a “gene gun”, inorganic chemical approaches such as calcium phosphate transfection and plasmid DNA incorporated into polymer coated stents. Ligand-mediated gene therapy, may also be employed involving complexity the DNA with specific ligands to form ligand-DNA conjugates, to direct the DNA to a specific cell or tissue.

The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondria genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

The term “vector” as used herein means a carrier that can contain or associate with specific nucleic acid sequences, which functions to transport the specific nucleic acid sequences into a cell. Examples of vector's include plasmids and infective microorganisms such as viruses, or non-viral vectors such as ligand-DNA conjugates, liposomes, lipid-DNA complexes. DNA sequence is operatively linked to an expression control sequence to form an expression vector capable of gene regulation. The transfected cells may be cells derived from the patient's normal tissue, the patient's diseased tissue (such as diseased vascular tissue), or may be non-patient cells. For example, blood vessel cells removed from a patient can be transfected with a vector capable of expressing a regulatory molecule of the present invention, and be re-introduced into the patient. Patients may be human or non-human animals. Cells may also be transfected by non-vector, or physical or chemical methods known in the art such as electroporation, incorporation, or via a “gene gun”. Additionally, DNA may be directly injected, without the aid of a carrier, into a patient.

The gene therapy protocol for transfecting a molecule into to patient may either be through integration of the molecule's DNA into the genome of the cells, into minichromosomes or as a separate replicating or non-replicating DNA construct in the cytoplasm or nucleoplasm of the cell. Modulation of gene expression and/or activity may continue for a transient period of time or may be reinjected periodically to maintain a desired level of gene expression and/or activity in the cell, the tissue or organ.

The present invention is further described by reference to the following non-limiting examples.

### EXAMPLE I

### Measuring Activin A, Activin B and Follistatin Levels in Patients Diagnosed with Chronic Fatigue Syndrome (CFS)

### Participants

Participants in this study included 47 patients (42 females and 5 males) diagnosed with chronic fatigue syndrome at the CFS Discovery Clinic, Melbourne, Australia. Patients were diagnosed with CFS if they fulfilled the Canadian Diagnostic CFS Criteria (Carruthers et al. “Myalgic encephalomyelitis/chronic fatigue syndrome: clinical working case definition, diagnostic and treatment guidelines. A consensus document.” J Chronic Fatigue Syndr 2003; 11: 7-115). The Canadian Diagnostic CFS Criteria have been found to identify more symptomatic patients with less concurrent psychiatric impairment when compared to other criteria (Jason et al. “Comparing the Fukuda et al. criteria and the Canadian case definition for chronic fatigue syndrome,” J Chronic Fatigue Syndr 2004; 12: 37-52).

### Task Procedure

A 20-min standing test was conducted. The task began with patients supine for 5 min after which they were instructed to stand upright and still, without support, for as long as possible, with the test capped at 20 minutes. Patients were encouraged to continue if they became fatigued. If patients discontinued the task, their total standing time was noted. Patients were asked to describe their fatigue. A 20 minute standing test was conducted under the supervision of a trained practice nurse. This test was found to have strong predictive relationships with functional fatigue levels. Patients were also asked to describe their fatigue level on a scale of 1-10 (1=no difficulty standing, 10=support required to stand, pre-syncope). For the purpose a this study, this standing difficulty scale was extended to 0-14, with a subjective or of 12 indicating standing difficulty to the point that the standing test was terminated at less than 20 minutes, and a score of 14 representing the most extreme difficulty where standing was only possible for 4-5 minutes, or less.

With the majority of the CFS/ME cohort achieving a standing time of 20 minutes, albeit with difficulty, direct comparisons of standing responses for CFS/ME and healthy control cohorts were not informative. To standardise the standing time in relation to subjective standing difficulty, and produce a single fatigue response variable, the time standing (maximum 20 minutes, measured at 2 minute intervals) and standing difficulty were combined to produce one measure called the “Weighted Standing Time” (WST). The WST was calculated by the following equation:

Weighted Standing Time (WST)=Time Standing×(1−(Difficulty)/14)

Included in the WST calculation were both CFS/ME cases (n=42) and healthy control participants (n=17) to produce a response (dependent variable) scale that represented the absence of CFS/ME symptoms through to severe CFS/ME symptoms, as assessed by standing time and subjective difficulty to stand during the orthostatic intolerance test. The WST was then used as a marker of CFS functional severity.

### Samples

Blood samples were taken from patients after the 20 min standing test and the plasma isolated. Plasma samples were analysed for concentrations of activin A, activin B and follistatin. Concentrations of activin A were determined using a two-site ELISA (Oxford Bio-innovations, Cherwell, Oxfordshire, UK) as previously published (Knight et al. 1996, J Endocrinol 148:267-279). This assay measures both free and follistatin-bound activin A dimers and has no significant cross-reaction with other activin isoforms, such as activin B. Activin B was measured by ELISA as previously described (Ludlow et al 2009, Clinical Endocrinol 71:867-873). Follistatin concentrations were determined using an extensively validated radioimmunology (O'Connor et al. 1999, Hum Reprod 14:827-832).

### Statistical Analyses

Regarding activin A, activin B and follistatin, an index of activin bioavailability was derived by calculating the activin A/folllistatin and activin B/follistatin ratio. Furthermore, activin A, activin B and follistatin concentrations in chronic fatigue syndrome (CFS) patients were compared to the normal ranges (NR group) for these proteins generated using 141 healthy adult volunteers (D. J. Phillips & D. M. de Kretser, unpublished observations). Comparisons between activin and follistatin concentrations in CFS patients and normal range values were made using Mann-Whitney test for non-parametric distributions. Data are presented as means±SEM.

Multiple linear regression was used to determine the prediction of activin B with regards to weighted standing time (WST). ANOVA analyses with Dunnet's post-hoc test was used to assess the differences in activin B levels relative to fatigue severity as calculated by WST.

### Results

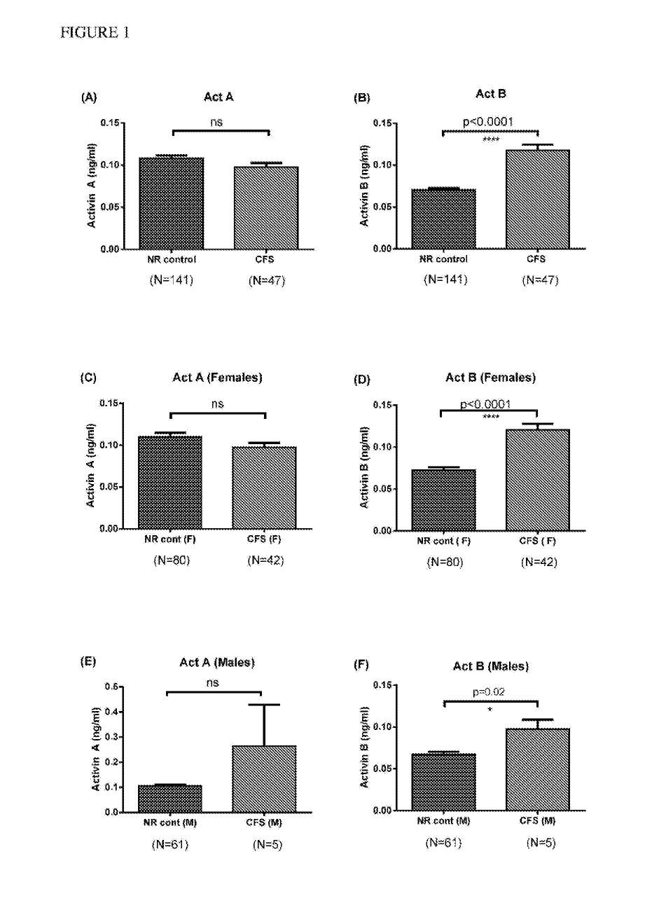

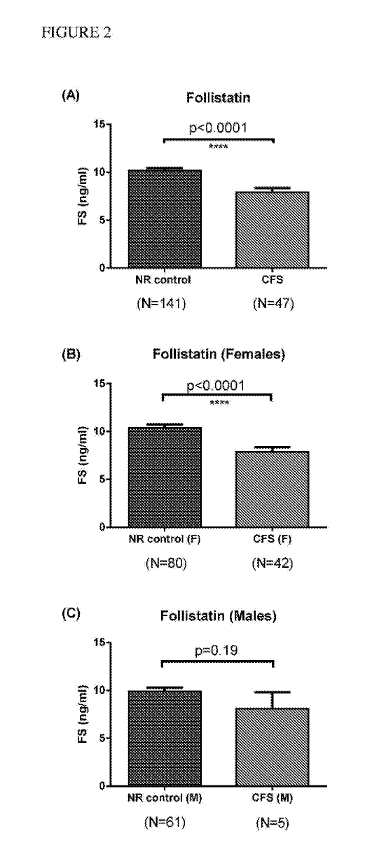

Activin B, but not activin A, levels were significantly higher in patients diagnosed with CFS compared to normal range (NR) group (FIG. 1). This elevation was seen in both male and female patients. Conversely, follistatin levels were significantly lower in the CFS group compared to the normal range group (FIG. 2). Analysis of male and female samples separately also showed that follistatin levels were significantly lower in female CFS patients. However, there was no significant difference observed in follistatin levels of male CFS patients compared to male normal controls, probably due to the low number of male CFS samples in this study.

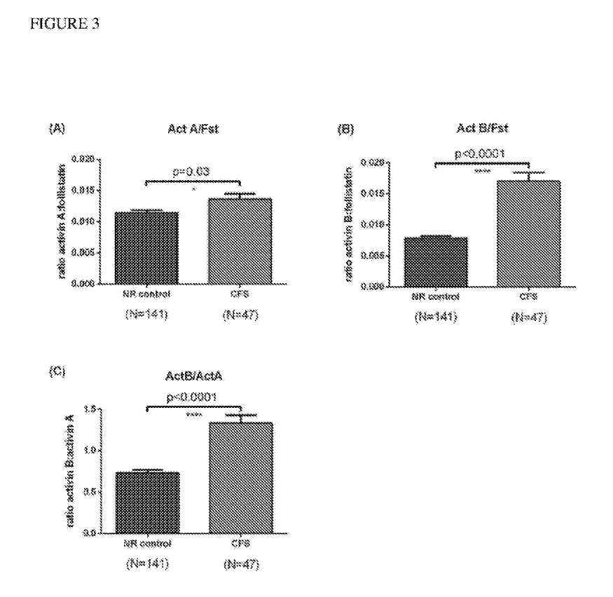

Comparisons of the activity to follistatin ratio showed that both activin A/follistatin and activin B/follistatin ratios were significantly higher in CFS patients than that seen in the normal range group (FIG. 3), with a greater difference seen for the activin B/follistatin ratio. Concordantly, activin B levels relative to activin A levels (activin B:activin A ratio) were significantly elevated in CFS patients compared to normal range group (FIG. 3).

This data shows that activin B levels and activin to follistatin ratios are elevated in people suffering from chronic fatigue syndrome.

### EXAMPLE 2

### Activin B Levels Relative to CFS Severity

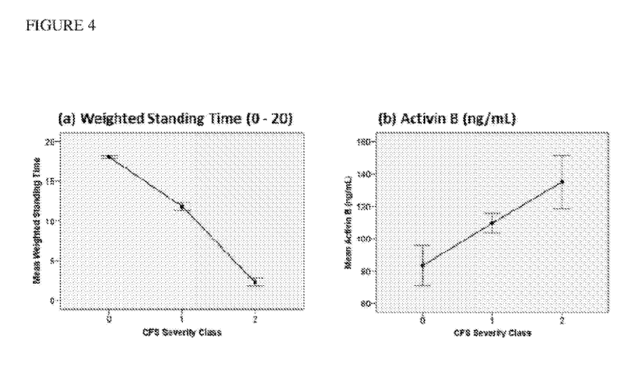

Multiple linear regression analysis identified that activin B was a significant predictor of the weighted standing time (WST) calculated for each CFS patient (p=0.013). Therefore, activin B levels relative to the WST was further assessed. The weighted standing time (WST) was divided into 3 categories: category 0 represented least severe CFS patients (n=2) and healthy controls (n=17)with WST values of 17.14-20.00 (all stood for 20 mins at difficulty 0-2); category 1 represented moderately severe CFS patients (n=30) with WST values of 7.14-157 (all stood for 20 mins at difficulty 3-9); and category 2 represented most severe CFS patients with WST values <=5.14 (all stood for <20 mins at difficulty 10-14). The data is shown in FIG. 4. WST and standing difficulty were significantly different between the three classes (p<0.001), with the shortest WST observed for the “most severe” class (FIG. 4a). Therefore, the WST was an excellent indicator of CFS severity.

Serum activin B levels were significantly elevated with increasing CFS severity (p=0.011), as determined via the WST response classes (FIG. 4b). By contrast, Activin A and follistatin were not found to be significant for a WST relationship, either through ANOVA or correlation/regression investigations.

Therefore, the data shows that activin B, but not activin A, is a possible diagnostic marker for CFS and also presents a good therapeutic target for the treatment of CFS.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bernard et al. 2004, Molecule Endocrinol 18:606-623

Bunin et al. 1994, Proc Nat Acad Sci USA 91:4708-4712

Carruthers et al. “Myalgic encephalomyelitis/chronic fatigue syndrome: clinical working case definition, diagnostic and treatment guidelines. A consensus document.” J Chronic Fatigue Syndr 2003; 11: 7-415

Jennifer N. Cash, Elizabeth B. Angerman, Henry T. Keutmann, and Thomas B. Thompson 2012, Characterization of Follistatin-Type Domains and Their Contribution to Myostatin and Activin A Antagonism. Mol Endocrinol, 26(7):1167-1178

Chalk et al. 2004, Biochem Biophys Res Commun 319:264-274

Conrad et al. 1995, Mol Divers 1:69-78

Ciu et al. 2004, Comput Methods Programs Biomed 75:67-73

DeWitt et al. 1993, Proc Natl Acad Sci USA 90:6909-6913

Ding, et al., Nucl Acids Res 32 Web Server issue, W135-W141

Ding & Lawrence 2003, Nucl Acids Res 31:7280-7301

Ding & Lawrence 2001, Nucl Acids Res 20:1034-1046

Elbashir et al. 2000, Methods 26:199-213

Elbashir et al. 2002, Methods 26:199-213

Ellington 1994, Curr Biol. 4:427-429

Harrison et al. 2004, J Biol Chem 279:28036-28044

Jason et al. “Comparing the Fukuda et al. criteria and the Canadian case definition for chronic fatigue syndrome.” J. Chronic Fatigue Syndr 2004; 12: 37-52

Henry T. Keutmann, Alan L. Schneyer and Yisrael Sidis 2004 The Role of Follistatin Domains in Follistatin Biological Action. Mol Endocrinol, 18(1):228-240

Khvorova et al. 2003, Cell 115:209-216

Klug et al. 1994, Mol Bio Rep 20:97-107

Knight et al. 1996, J Endocrinol 148:267-279

Kohler and Milstein 1975, Nature 256:495-499

Kohler and Milstein 1976, Eur J Immun 6:511-519

Lato et al. 1995, Chem Biol 2:291-303

Ludlow, H., et al., (2009) “A new ‘total’ activin B enzyme-linked immunosorbent assay (ELISA): development and validation for human samples.” Clinical Endocrinology 71: 867-873

Ludlow et al 2009, Clinical Endocrinol 71:867-873

Maeshima et al. 2004, Endocrinology 145:3739-3745

Makanji Y et al. 2011 Generation of a specific activin B antagonist by modification of the activin B propeptide. Endocrinol 152:3758-3768

Moore, A., Basilion, J., Chiocca, E., and Weissleder, R., BBA, 1402:239-249, 1988

O'Connor et al. 1999, Hum Reprod 14:827-832

Poulaki et al. 2004. Am J Pathol 164:1293-1302

Reynolds et al. 2004, Nat Biotechnol. 22:326-330

Schwarz, et al. 2003, Cell 115:199-208

Stein and Cohen, 1988, Cancer Res 48:2659-2668

Takabe et al. 2003, Hepatology 38:1107-1115

Uphoff et al. 1996, Curr Op in Struct Biol 6:281-287

van der Krol et al., 1988, Biotechniques 6:958-976

Wallis et al. 1995, Chem Biol 2:543-552

Wang et al. 2004, Bioinformatics 20:1818-1820

Wedemeyer, N., Potter, T., Wetzlich, S. and Gohde, Clinical Chemistry 48:9 1398-1405, 2002

Weissleder, R., Moore, A., Ph.D., Mahmood-Bhorade, U., Benveniste, H., Chiocca, E. A., Basilion, J. P. Nature Medicine, 6:351-355, 2000

Xu et al. 1995 J Biol Chem 270:6308-6313

N. Yang, Gene Transfer into Mammalian Somatic Cells in vivo, Crit. Rev. Biotech. 12(4):335-356 (1992)

1.-25. (canceled)

26. A method for detecting chronic fatigue syndrome in a mammal, comprising screening for the level of activin β_{B} protein and or/gene expression in said mammal or in a biological sample derived from said mammal, wherein an increase in the level of said protein and/or gene expression relative to normal levels is indicative of chronic fatigue syndrome.

27. The method according to claim 26, wherein said activin β_{B} is in monomeric form or homodimeric form.

28. A method of detecting chronic fatigue syndrome in a mammal, comprising screening for the level of one or more of:
(i) activin B:follistatin protein and/or gene expression ratio;
(ii) activin B:activin A protein and/or gene expression ratio;
(iii) activin β_{B}:follistatin protein and/or gene expression ratio;
(iv) activin β_{B}:activin A protein and/or gene expression ratio; or
(v) activin β_{B}:activin β_{A} protein and/or gene expression ratio;
in said mammal or in a biological sample derived from said mammal, wherein an increase in the level of said ratio relative to normal levels is indicative of chronic fatigue syndrome.

29. A method for monitoring the progression of chronic fatigue syndrome in a mammal, comprising screening for modulation of the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(vii) activin β_{B}:activin β_{A} protein and/or gene expression ratio;
in said mammal or in a biological sample derived from said mammal, wherein an increase in the level or ratio of said protein and/or gene expression relative to a previously obtained level is indicative of worsening of said condition, a decrease in said level is indicative of an improvement in said condition, and no change to said level is indicative of no significant change to the severity of said condition.

30. The method according to claim 29, wherein said method comprises screening for modulation for one or more of:
(i) activin β_{B} protein and/or gene expression;
(ii) activin B protein and/or gene expression;
(iii) activin B:follistatin protein and/or gene expression ratio; or
(iv) activin B:activin A protein and/or gene expression ratio.

31. A method of assessing the severity of chronic fatigue syndrome in a mammal, said method, comprising determining the level of one or more of:
(i) activin β_{B} protein and/or gene expression;
(ii) activin B protein and/or gene expression;
(iii) activin B:follistatin protein and/or gene expression ratio;
(iv) activin B:activin A protein and/or gene expression ratio;
(v) activin β_{B}:follistatin protein and/or gene expression ratio;
(vi) activin β_{B}:activin A protein and/or gene expression ratio; or
(vii) activin β_{B}:activin β_{A} protein and/or gene expression ratio;
in said mammal or in a biological sample derived from said mammal, wherein the higher the level or ratio of said protein and/or gene expression then the more severe the chronic fatigue syndrome.

32. The method according to claim 31, wherein said method comprises screening for the modulation of one or more of:
(i) activin β_{B} protein and/or gene expression;
(ii) activin B protein and/or gene expression;
(iii) activin B:follistatin protein and/or gene expression ratio; or
(iv) activin B:activin A protein and/or gene expression ratio.

33. The method according to claim 26, wherein said protein comprising the β_{B} subunit is activin B.

34. The method according to claim 26, wherein said method is directed to screening for:
(i) activin B, activin A or follistatin protein, or
(ii) activin B, activin A or follistatin mRNA.

35. The method according to claim 26, wherein the mammal which is screened is a mammal exhibiting one or more symptoms selected from the group consisting of:
(i) post-exertional malaise;
(ii) neuro-cognitive problems;
(iii) disrupted sleep;
(iv) pain or aches in the muscles, joints or head;
(v) a drop in blood pressure, feeling dizzy or pale;
(vi) palpitations, increased heart rate, or shortness of breath with exertion or on standing;
(vii) allergies or sensitivities to light, smells, touch, sound, foods, chemicals and medications;
(viii) gastrointestinal changes;
(ix) urinary problems;
(x) sore throat, tender lymph nodes and a flu-like feeling;
(xi) marked weight change;
(xii) inability to cope with temperature changes;
(xiii) brain fog;
(xiv) difficulty maintaining an upright position, dizziness, balance problems or fainting;
(xv) allergies or sensitivities to foods, odors, chemicals, medications, or noise;
(xvi) symptoms of irritable bowel syndrome;
(xvii) chills and night sweats;
(xviii) visual disturbances; and
(xix) depression or mood problems.

36. A method of treating chronic fatigue syndrome in a mammal, comprising downregulating the functional activity of activin B in said mammal.

37. The method according to claim 36, wherein the mammal which is the subject of treatment is exhibiting one or more symptoms selected from the group consisting of:
(i) post-exertional malaise;
(ii) neuro-cognitive problems;
(iii) disrupted sleep;
(iv) pain or aches in the muscles, joints or head;
(v) a drop in blood pressure, feeling dizzy or pale;
(vi) palpitations, increased heart rate or shortness of breath with exertion or on standing;
(vii) allergies or sensitivities to light, smells, touch, sound, foods, chemicals and medications;
(viii) gastrointestinal changes;
(ix) urinary problems;
(x) sore throat, tender lymph nodes and a flu-like feeling;
(xi) marked weight change;
(xii) inability to cope with temperature changes;
(xiii) brain fog;
(xiv) difficulty maintaining an upright position, dizziness, balance problems or fainting;
(xv) allergies or sensitivities to foods, odors, chemicals, medications, or noise;
(xvi) symptoms of irritable bowel syndrome;
(xvii) chills and night sweats;
(xviii) visual disturbances; and
(xix) depression or mood problems.

38. The method according to claim 36, wherein said activin B is downregulated by administering an activin B antagonist.

39. The method according to claim 38, wherein said activin B antagonist is selected from the group consisting of:
(i) follistatin;
(ii) the a subunit of inhibin;
(iii) inhibin;
(iv) antibody directed to activin B or the activin β subunit;
(v) a non-functional activin mutant;
(vi) a non-functional activin B receptor mutant;
(vii) a soluble activin B receptor;
(viii) an activin B antisense oligonucleotide;
(ix) a thrombin antagonist;
(x) the Cripto protein;
(xi) an inhibitor of the ALK7 or ALK3 receptor;
(xii) an activin β_{B} antisense oligonucleotide;
(xiii) a DNAzyme;
(xiv) an aptamer; and
(xv) molecules suitable for use in co-suppression of activin expression.

40. The method according to claim 39, wherein said follistatin is FS315 or FS288.

41. The method according to claim 39, wherein said follistatin is selected from the group consisting of:
(i) wild-type follistatin (FS), comprising an N-terminal domain (ND) followed by three follistatin domains (FSD1, FSD2 and FSD3) with a heparin-binding sequence located in FSD1 (amino acid sequence positions 72-86), and all known isoforms thereof;
(ii) wild-type follistatin-like 3 protein (FSTL3) (also known as follistatin-related gene product (FLRG) and follistatin-related protein (FSRP)), comprising an N-terminal domain (N3D) followed by two follistatin-like 3 domains (FS3D1 and FS3D2), and all known isoforms thereof;
(iii) follistatin analogs having the structure ND-FSD1-FSD2;
(iv) analogs of (i) and (iii) wherein FSD1 is substituted by FSD1′, where FSD1′ represents FSD1 with its heparin-binding site removed;
(v) analogs of (i) and (iii) above wherein FSD1 is substituted by FSD1*, where FSD1* represents FSD1 with a sequence prior to and including its heparin-binding sequence removed;
(vi) hybrid forms of (i) and (iii) above wherein at least one of the domains is substituted by a corresponding FSTL3 domain N3D, FS3D1 and FS3D2;
(vii) hybrid forms of (ii) above wherein at least one of the domains is substituted by a corresponding FS domain ND, FSD1, FSD1′, FSD1* and FSD2;
(viii) any of the above proteins modified by one or more deletions, insertions and/or mutations in ND, N3D, FSD1, FSD1′, FSD1*, FS3D1, FSD2, FS3D2, and FSD3, provided the modified protein functions as an activin B antagonist; and
(ix) genetically modified forms of follistatin which have been modified to preferentially antagonize activin B over other activin or follistatin targets.

42. The method according to claim 36, wherein said treatment is therapeutic or prophylactic.

43. The method according to claim 26, wherein said mammal is a human.
